(19) United States
(12) Patent Application Publication
(10) Pub. No.: US 2015/0291663 A1
(43) Pub. Date: Oct. 15, 2015

(54) NEW PEPTIDES AS A MONOTHERAPY IN MALIGNANCY CONTROL

(71) Applicant: Khaled Mohey Eldin Elawdan, Mehalia El-Kobra (EG)

(72) Inventor: Khaled Mohey Eldin Elawdan, Mehalia El-Kobra (EG)

(21) Appl. No.: 14/422,358

(22) PCT Filed: Apr. 9, 2013
(86) PCT No.: PCT/EG2013/000008

§ 371 Date: Feb. 19, 2015

(51) Int. Cl. C07K 14/00; C07K 7/08

(52) U.S. Cl. CPC C07K 14/00; C07K 7/08

(57) ABSTRACT

Peptides with sequences either in whole or in part or similar sequences as a monotherapy for cancer control, that is, without any need for conventional methods except for diagnosis. Any one or more of the peptides from N Terminus to C Terminus can be used as a drug in malignancy control and management.

### TECHNICAL FIELD

New Peptides as A monotherapy in Malignancy Control

### BACKGROUND ART

We have four main lines in management of malignancy.

Surgery: Surgery can be used to prevent, treat, stage (determine how advanced the cancer is), and diagnose cancer. In relation to cancer treatment, surgery is done to remove tumors or as much of the cancerous tissue as possible. It is often performed in conjunction with chemotherapy or radiation therapy.

For those whose cancer is not treatable, palliative surgery may be an option to relieve pain that may be caused by the cancer. Palliative surgery is not intended to treat or cure the cancer, or even to prolong life, but more to lessen discomfort.

Chemotherapy: Chemotherapy is a type of cancer treatment that uses of drugs to eliminate cancer cells. Unlike surgery, chemotherapy affects the entire body, not just a specific part. It works by targeting rapidly multiplying cancer cells. Unfortunately, other types of cells in our bodies also multiply at high rates, like hair follicle cells and the cells that line our stomachs. This is why chemo can cause side effects like hair loss and an upset stomach.

Chemotherapy is most commonly given by pill or intravenously (IV), but can be given in other ways. A single type of chemotherapy, or a combination of drugs, may be prescribed for a specific length of time. Like surgery, chemotherapy can be prescribed alone, in conjunction with radiation therapy or biologic therapy.

Radiation Therapy: Radiation therapy uses certain types of energy to shrink tumors or eliminate cancer cells. It works by damaging a cancer cell's DNA, making it unable to multiply. Cancer cells are highly sensitive to radiation and typically die when treated. Nearby healthy cells can be damaged as well, but are resilient and are able to fully recover.

Radiation therapy may be given alone, along with chemotherapy, and/or with surgery. The decision to combine radiation therapy with other types of treatment depends on the stage of cancer and other factors

Biologic or Targeted Therapy:

Biologic therapy is a term for drugs that target characteristics of cancerous tumors. Some types of targeted therapies work by blocking the biological processes of tumors that allow tumors to thrive and grow. Other types of therapies cut off the blood supply to the tumor, causing it to basically starve and die because of a lack of blood.

Targeted therapy is used in select types of cancer and is not available for everyone. It is given in conjunction with other cancer treatments.

Problem and Defect:

Although of many methods in management of malignancy like surgery, chemotherapy, radiotherapy, immunotherapy . . . etc.

Many types of cancer still have very poor prognosis.

It is enough for anybody to have a look at the main causes of death in any country to know that cancer is one of them and to discover how the challenge and defect in its treatment.

Many researches aim to just improvement of the already present methods.

Anyhow, I think we must introduce something new rather than just thinking of improvement of traditional methods to overcome this challenge.

### DISCLOSURE OF INVENTION

We introduce those peptides as a monotherapy in malignancy control:

Either one of those peptides or more than one till all with these sequences from N Terminus to C terminus.

1-MAILTYVYVF AVLFIANSMQ A
2-MAILTYVYVF AVLFIAKKKK
3-MQLRVLFFFL FVATISYAIA D
4-MQLRVLFFFL FVATISKKKKK
5-MQLKALFFLL FAATISKKKKK
6-MTKWLLLVVC LGIACQ
7-MTKWLLLVVC LGIACQKKKKK

So, we ask protection of those peptides sequences as it can be used either in one or more as a drug in cancer control.

1. To avoid any biased data, we confirmed our primary results in another far research company—Genscript USA—as a paid business.

2. Those are our unbiased data sent to us by Genscript.

3. 6 peptides were sent for efficacy studies against 15 tumor cell line (in vitro) studies.

4. 3 peptides were not in a good condition with a very low purity, so the study was limited to only 3 peptides, all with purity of more than 95%; peptide A, F & F with those sequences:
A:MAILTYVYVF AVLFIAKKKK
E:MQLRVLFFEL FVATISKKKKK
F:MTKWLLLVVC LGIACQKKKKK

5. 4 groups were designed; one for peptide A, 2 for peptide E, 3 for peptide F and 4 for peptides A+E+F with 100 ug of each peptide was used in each group.

6. TFA 1% was used as a control as we used lyophilized peptides in 1% TFA salt.

7. The results showed very high inhibitory effects of group 4 (A+E+F) then group 3 (F) then group 1(A) and lastly group 2 (E) upon most tested tumor cells (final report was attached).

8. As a preparation for in vivo studies, we did another 2 studies for certain 4 tumor cells that we are going to use in our in vivo studies.

9. For those 4 tumor cells, peptides(A+F only as we exclude E) were tested for apoptosis using 50 ug of each peptide as well as efficacy with increasing doses of peptides starting from 10 ug, 25 ug, 50 ug, 75 ug and 100 ug of each peptide (final report was attached).

10. for those peptides:
B-MAILTYVYVF AVLFIANSMQ A
C-MQLRVLFFFL FVATISYAIA D
D-MTKWLLLVVC LGIACQ
We did discover inhibitory effects upon C6, U251 and UW3 with the best inhibitory effects to D peptide then B peptide and lastly C peptide (crude purity peptides). The problem was that, those peptides were very hydrophobic and with our trials with many biotechnology companies to synthesize, they failed to get a high purity except crude with a purity of only around 70%.
Only one company claimed its ability to produce those peptides with a purity of more than 95% as we asked, but we discovered that this was not true and as well later on, Genscript company staff confirmed that, when we sent all peptides for confirmation of our primary results.

11. for peptide:
G-MQLKALFFLL FAATISKKKKK
It is similar in structure as well as effects to E peptide (MQLRVLFFFL FVATISKKKKK).


SEQUENCE LISTING (7 sequences)

SEQ ID NO: 1 - protein, 21 aa, artificial sequence
  synthetic peptide
MAILTYVYVFAVLFIANSMQA

SEQ ID NO: 2 - protein, 20 aa, artificial sequence
  synthetic peptide
MAILTYVYVFAVLFIAKKKK

SEQ ID NO: 3 - protein, 21 aa, artificial sequence
  synthetic peptide
MQLRVLFFFLFVATISYAIAD

SEQ ID NO: 4 - protein, 21 aa, artificial sequence
  synthetic peptide
MQLRVLFFFLFVATISKKKKK

SEQ ID NO: 5 - protein, 21 aa, artificial sequence
  synthetic peptide
MQLKALFFLLFAATISKKKKK

SEQ ID NO: 6 - protein, 16 aa, artificial sequence
  synthetic peptide
MTKWLLLVVCLGIACQ

SEQ ID NO: 7 - protein, 21 aa, artificial sequence
  synthetic peptide
MTKWLLLVVCLGIACQKKKKK